(19) United States
(12) Patent Application Publication
(10) Pub. No.: US 2012/0266367 A1
(43) Pub. Date: Oct. 25, 2012

(54) GOGGLE STRAP SYSTEM

(76) Inventor: Brent Sandor, Encinitas, CA (US)

(21) Appl. No.: 13/090,023

(22) Filed: Apr. 19, 2011

(51) Int. Cl. A61F 9/02

(52) U.S. Cl. 2/431

(57) ABSTRACT

A sports goggle with a strap system that is convenient to engage as well as adjust is disclosed. The strap system includes first and second straps which extend from left and right end portions of the frame of the goggle. The first strap may be fed through an attachment member attached to a distal end of the second strap. To set the tension in the first and second straps, the first strap is pulled until the tension in the first and second straps has reached the desired level. This tension adjustment occurs while the goggle is on the participant's head. Once the desired tension level is reached, hooks and loops on the first strap are engaged to each other to set the tension level.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

The embodiments disclosed herein relate to a sports goggle.

Participants of a sport or activity (e.g., motocross or snow skiing/snowboarding) may wear a goggle to protect the participant's eyes from the natural elements. For example, a snow goggle is typically worn about a participant's eyes to protect the participant's eyes from the elements (e.g., snow, wind, etc.) while participating in a snow activity. By way of example and not limitation, while snowboarding or skiing down a snow covered mountain, the participant's eyes are exposed to cold wind, bright sunlight, reflections of the sunlight and snow. As the snow participant is traversing down the mountain side, cold wind flows past the participant's eyes. The constant flow of cold air over the participant's eyes dries out the participant's eyes over the course of the day. Moreover, after a snow storm when the clouds have cleared the sky, the participant is exposed to bright sunlight as well as reflections of the sun off of the white snow. The participant is also subjected to snow if the participant is skiing or snowboarding during snow fall. The snowflakes enter the participant's eyes and irritate the participant's eyes so as to distract the participant from being fully aware of his/her surroundings. Additionally, the participant may fall down and have snow sprayed in his/her face. The snow goggle provides a lens which diverts cold wind away from the participant's eyes. The lens may also be tinted to reduce strain on the participant's eyes due to the bright sun and/or reflections of the sunlight on the white snow. The lens also protects the participant's eyes since any snowflake or snow projected toward the participant's eyes will be blocked by the lens. Accordingly, the goggle is an important piece of equipment to enjoy the snow activity.

A motorcycle rider will also wear a goggle to protect his/her eyes from mud, dirt, dust, rain, sunlight and reflections of the sunlight. For example, in a group, the riders in the front will spew up dirt, dust and mud. The riders in the rear must ride through the dirt, dust and mud. Additionally, during a bright sunny day, the rider's eyes will be exposed to the bright sun as well as reflections of the sunlight off of the ground and other objects. The motocross goggle protects the rider's eyes from the dirt, dust and mud by providing a lens disposed in front of the rider's eyes. The lens also diverts wind away from the rider's eyes to prevent dryness of the eyes during participation of the sport. The lens may also be tinted to reduce strain on the rider's eyes due to the bright sun or reflections of the sunlight on various objects. Accordingly, the motocross goggle is an important piece of equipment for the enjoyment of riding a motorcycle.

Prior art sports goggles are designed to fit over a helmet. Since the goggles are generally one size fits all, the goggles have adjustable straps that may be adjusted by a friction based strap system. Although the straps may be adjusted while the participant is still wearing gloves or mittens, the adjustment mechanism is typically manipulated while the participant is not wearing his/her gloves or mittens. Moreover, the strapping system once set is difficult to readjust because the strap system is designed to stay in place despite vigorous use throughout the day. In order to adjust the strap tension of the goggle, the fitting process is typically an iterative trial and error process. The participant estimates the appropriate strap length and then tries on the goggle. Based on the fit, the participant removes the goggle and readjusts the strap system to either loosen or tighten the straps. The participant then tries on the goggle again to determine whether the tension in the straps is appropriate. The participant goes through this trial and error process until the tension in the straps is at the appropriate level. Although the fitting process could be performed while the goggle remains on the participant's head, the fitting process is typically accomplished by taking the goggle on and off as described above due to the difficulty of adjusting the strap system while the goggle is still on the participant's head. When the outside ambient temperature is cold, this trial and error process is typically done indoors since the strap system is manipulated typically while the participant is not wearing gloves. If this process was performed outdoors, then the participant's hands would be uncomfortably exposed to the cold air. The fitting process could be performed while the participant is still wearing his/her gloves or mittens but it is inconvenient to do so. If the straps must be adjusted while participating in the sport (e.g., snow activity or motocross), the user must stop and remove his/her gloves which is time consuming and slows down the pace of the participant.

Accordingly, there is a need in the art for an improved strap system for a sports goggle.

### BRIEF SUMMARY

The sports goggle discussed herein addresses the needs discussed above, discussed below and those that are known in the art.

The sports goggle has a lens that covers the participant's eyes to protect the participant from wind, snow, mud, dirt, dust, rain and may also be semi transparent to protect the participant's eyes from bright sunlight as well as reflections of the sunlight off of the white snow, ground, cars, or other reflective surfaces. The sports goggle is secured to the participant's head through a strap system which is convenient to attach as well as adjust. The strap system allows the participant to conveniently manipulate the straps. Additionally, to adjust the tension in the straps, no trial and error or iterative process is required. The participant merely pulls on the first strap until the desired tension is reached then secures the first strap to set the tension of the straps. More particularly, first and second straps extend from left and right end portions of the frame of the goggle. The first strap has a latch mechanism such as hooks and loops which can be removably engageable to each other. The second strap has an attachment member which is sized and configured to receive the first strap. To secure the goggle to the participant's head, the first strap is fed through the attachment member. The participant pulls on the first strap until the desired tension in the first and second straps is reached. Once the desired tension in the straps is reached, the hooks and loops are engaged to each other to set the tension in the first and second straps. It is contemplated that the entire adjustment process can be accomplished while the participant is wearing his/her gloves or mittens.

More particularly, a sports goggle for viewing a person's surroundings and preventing particulate (e.g., snow, mud, dirt, dust, etc.) from contacting the person's eyes during a sports activity is disclosed. The goggle may comprise a lens, a frame, a first strap, and a second strap. The lens may be disposed in front of the person's eyes. The frame may hold the lens and have a soft cushion which interfaces with the person's face. The frame also defines opposed left and right end portions. The first strap may be attached to one of the left or right end portions of the frame. A distal end portion of the first strap may have first and second parts of a latch. The second strap may be attached to the other one of the left or right end portions of the frame. A distal end portion of the second strap may have an attachment member. The distal end portion of the first strap may be fed through the attachment member until the tension in the straps is at the desired level. The first part of the latch may be engaged to the second part of the latch for securing the second strap and the first strap together at a fixed tension.

The first and/or second straps may be elastic. It is contemplated that the entire or at least a portion of the first and/or second straps be elastic.

The first and second parts of the latch may be hooks and loops, loops and hooks, snaps or a button/button holes. If the latch is hooks and loops or vice versa, then the hooks and loops may be disposed on an outer side of the second strap.

The lens may be a unitary lens that covers both the left and right eyes of the wearer or separate left and right lenses that covers the left and right eyes respectively.

Additionally, a sports goggle for viewing a person's surroundings and preventing particulate (e.g., snow, mud, dirt, dust, etc.) from contacting the person's eyes during a sports activity is disclosed. The goggle may comprise a lens, a frame, a first strap and an attachment member. The lens may be disposed in front of the person's eyes. The frame may hold the lens and have a soft cushion which interfaces with the person's face. The frame also defines opposed left and right end portions. The first strap may be attached to one of the left or right end portions of the frame. A distal end portion of the first strap may have first and second parts of a latch. The attachment member may be attached to the other one of left or right end portions of the frame. The distal end portion of the first strap may be fed through the attachment member and the first part of the latch engaged to the second part of the latch for securing the first strap to the frame.

At least a portion of the first strap may be elastic.

The first and second parts of the latch may be hooks and loops, loops and hooks, snaps or a button and button holes. If the latch is hooks and loops or vice versa, the hooks and loops may be disposed on an outer side of the first strap.

Moreover, a method for securing a goggle to a person's head is disclosed. The method may comprise the steps of providing a goggle with a first strap attached to one of a left or right end portion of a goggle frame wherein the first strap is fed through an attachment member attached to the other one of the left or right end portion of the goggle frame for adjusting tension in the first strap; adjusting tension in the first strap by feeding more or less of the first strap through the attachment member; and engaging a first part of a latch on the first strap to a second part of the latch on the first strap for securing the first strap to the attachment member.

The method may further comprise the step of disposing the goggle around the person's head.

The method may further comprise the step of disposing a sports helmet on the person's head over the first strap.

The providing step may comprise the step of providing the attachment member on a second strap wherein the second strap is attached to the other one of the left or right end portions of the frame.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a front perspective view of a sports goggle with straps indirectly attached to a frame of the goggle;

FIG. 2 is a rear perspective view of the sports goggle shown in FIG. 1;

FIG. 3 is a front view of the sports goggle shown in FIG. 1 with first and second straps disengaged from each other; and

FIG. 4 is a front perspective of the sports goggle with straps directly attached to the frame of the goggle.

### DETAILED DESCRIPTION

Referring now to the drawings, a sports goggle 10 that may be worn around the person's head is shown. The sports goggle 10 allows the person to view his/her surrounding while preventing objects such as snowflakes, mud, dirt, dust, etc. from hitting the person's eyes so that the person may maintain visual awareness of his/her surroundings. The sports goggle 10 is conveniently attachable to the person's head by way of first and second straps 12, 14 that are removably attachable to each other. The tension in the straps 12, 14 is also easily adjustable. The first strap 12 may be fed through an attachment member 16 (e.g., ring, buckle, or any other shape or style, etc.) attached to the second strap 14. The tension in the first and second straps 12, 14 may be adjusted by feeding more or less of the first strap 12 into the attachment member 16. The sports goggle 10 described herein allows the person to conveniently adjust the strap tension without trial and error. The first strap 12 is fed through the attachment member 16 and while the goggle 10 is on the person's face, the person pulls on the distal end portion of the first strap 12 in direction of arrow 50 until the tension in the first and second straps 12, 14 has reached the desired level. Once the tension in the first and second straps 12, 14 is at the desired level, the first strap is attached to itself (e.g., hooks and loops, snaps, buttons, etc.) by pivoting the first strap 12 in direction of arrow 54 to fix the tension in the first and second straps. The participant is now ready to enjoy the physical activity (e.g., snow skiing, snowboarding, motocross, etc.).

More particularly, the sports goggle 10 may have a frame 18 that holds a clear transparent lens 20 (e.a., tinted or clear). The transparent lens 20 is shown as being a unitary transparent lens, but it is also contemplated that the lens may be separated wherein a left lens covers a left eye of the person and a right lens covers a right eye of the person. The frame 18 holds the left and right lenses in place. The frame 18 additionally includes a cushioning member 22 which circumscribes the entire frame 18. The cushioning member 22 engages the person's face and circumscribes the eyes of the person. The frame 18, lens 20 and the cushioning member 22 provide a physical barrier so that snowflakes, mud, dirt, dust and other objects do not enter the person's eyes and distract the person while participating in a physical activity such as snow skiing, snowboarding or motocross. By way of example and not limitation, when a snow participant is traversing down a mountain, falling snowflakes will contact the eyes of the person. In this event, the person will blink or squint to avoid such uncomfortable contact. Moreover, during high speeds, cold snowflakes are extremely uncomfortable. Also, during snow skiing or snowboarding, the person may fall and have snow thrown into his/her face thereby irritating the eyes and face of the person. With the frame 18, lens 20 and cushioning member 22 disposed about the person's eyes, the person's eyes are protected from wind, snow and weather elements. The same is true for a motorcycle rider. Mud, dirt and dust are being thrown up in the air by riders in front. With the frame 18, lens 20 and cushioning member 22 disposed about the rider's eyes, the rider's eyes are protected from the wind, mud, dirt and dust.

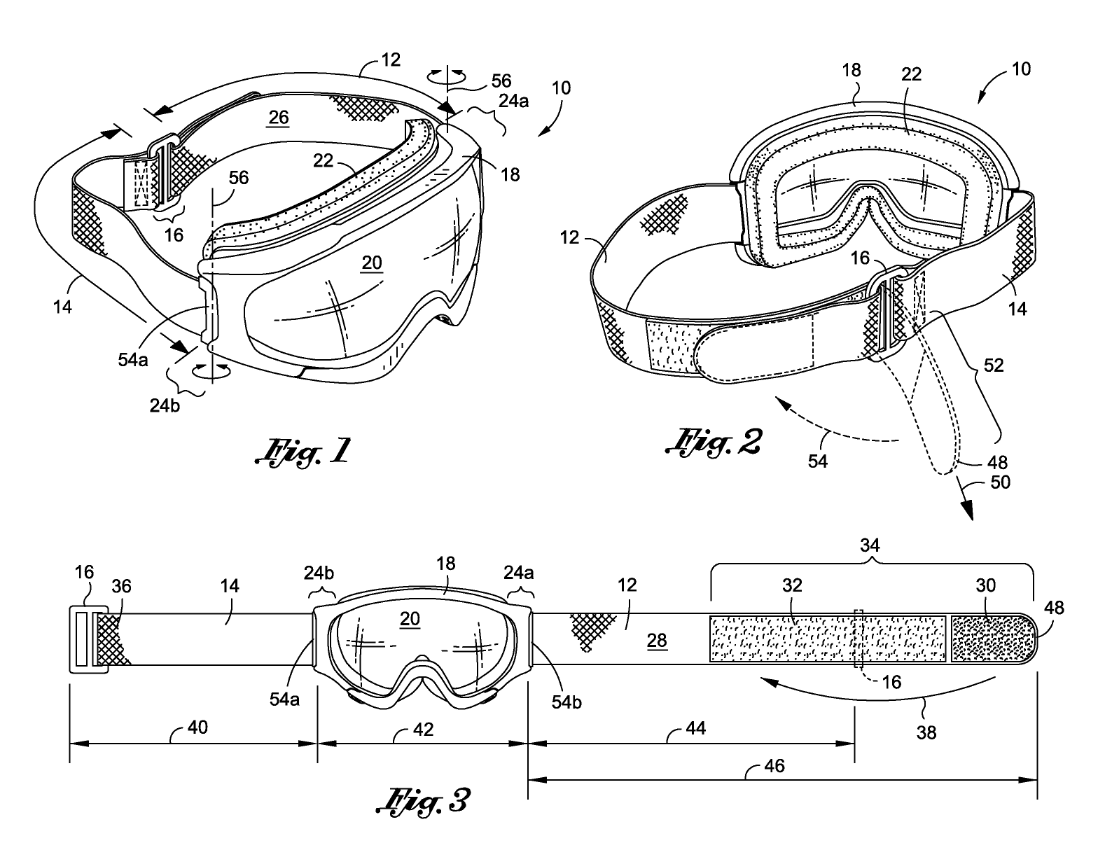

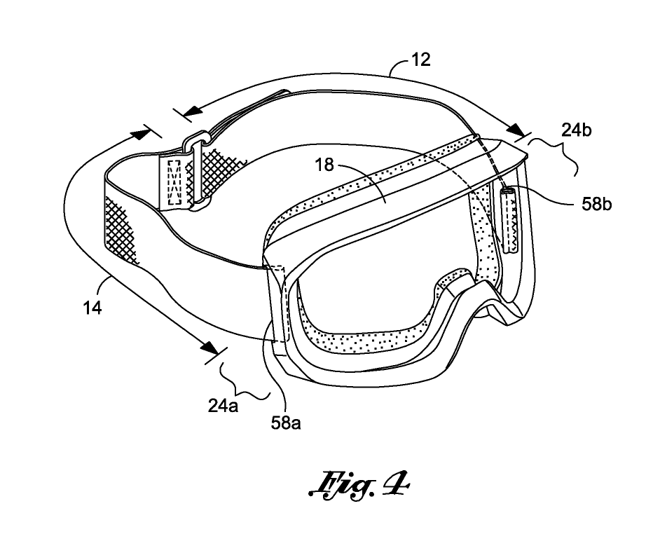

The frame 18 of the sports goggle 10 defines left and right end portions 24a, b. The first and second straps 12, 14 are respectively attached to the left and right end portions 24a, b of the frame 18. The drawings show the first strap 12 attached to the left end portion 24a and the second strap 14 attached to the right end portion 24b. However, it is also contemplated that the first strap 12 may be attached to the right end portion 24b and the second strap 14 may be attached to the left end portion 24a. For the purpose of clarity, only the embodiment where the first and second straps 12, 14 are respectively attached to the left and right end portions 24a, b of the frame 18 will be discussed. The first and second straps 12, 14 may be attached to the left and right distal end portions 24a, b by various known methods known in the art or developed in the future. In FIG. 1, the first and second straps 12, 14 are indirectly attached to the left and right end portions 24a, b of the frame 18 with internal outriggers 54a, b which pivots about pivot axis 56. Alternatively, the first and second straps 12, 14 may be directly attached to the left and right end portions 24a, b with slots 58a, b formed in the left and right end portions 24a, b of the frame 18, as shown in FIG. 4. The first and second straps 12, 14 are fed through the slots 58a, b and sewn to prevent pull out of the first and second straps 12, 14 so that the straps are permanently secured to the first and second distal end portions 24a, b of the frame 18. The straps 12, 14 extend horizontally away from the left and right end portions 24a, b as shown in FIG. 3. The first strap 12 defines inner and outer sides 26, 28 (see FIGS. 1 and 3). The outer side 28 of the first strap 12 may have first and second parts 30, 32 of a latch 34. The first and second parts 30, 32 may be hooks and loops or vice versa, first and second parts of a snap 34, a button and button holes or vice versa, or other latching mechanism. For example, one first part of the snap may be secured to the first strap 12. A plurality of second parts of the snap may be located longitudinally along the length of the first strap 12 so that the first part may be snapped to anyone of the second parts of the snaps depending on the desired tension in the first and second straps 12, 14. With respect to the hooks and loops 30, 32, the hooks 30 may be attached to any part of the loops 32 depending on the desired tension in the first and second straps 12, 14. The attachment member 16 may be attached to the distal end 36 of the second strap 14.

In use, the first and second straps 12, 14 are wrapped around the person's head. The distal end of the first strap 12 is fed through the attachment member 16 as shown in FIGS. 1 and 2. The first strap 12 is folded upon itself as shown in FIG. 2 so that the hooks 30 are attached to the loops 32. The hooks and loops may be attached to each other after the length of the first strap 12 and the tension in the straps 12, 14 are adjusted by feeding more or less of the first strap 12 through the attachment member 16. The person may now enjoy the outdoor activity (e.g., snowboarding, snow skiing, snow mobiling, motocross, etc.).

Referring now to FIG. 3, the attachment member 16 may be positioned anywhere along the first strap 12 on the loops 32 by feeding more or less of the first strap 12 into the attachment member 16 so long as the hooks 30 are engageable to the loops 32 when folded in the direction of the arrow 38, 54. The length 40 of the second strap 14, the length 42 of the frame 18 and the adjusted length 44 of the second strap 14 which is defined as the distance from the left end portion 24a of the frame to the position of the attachment member 16 on the first strap 12 defines the circumference. The circumference may be adjusted by adjusting the adjusted length 44 of the second strap 14 by feeding more of less of the first strap 12 through the attachment member 16 by pulling in the direction of arrow 50. The length 46 of the first strap 12 should be long enough to allow the first strap 12 to be fed through the attachment member 16 and wrap around the attachment member 16 and allow the hooks 30 to engage the loops 32 or to allow first and second parts of the latch to engage each other.

The first and second straps 12, 14 may be fabricated from an elastic material. It is also contemplated that one of the first and second straps 12, 14 may be fabricated from an elastic material while the other one of the first and second straps 12, 14 may be fabricated from a non-elastic material. Moreover, it is contemplated that only a portion of either one or both of the first and second straps 12, 14 is fabricated from an elastic material.

The hooks 30 are shown as extending to the distal end 48 of the first strap 12. However, it is also contemplated that the distal end 48 may have a relatively hard substance (e.g., leather reinforced member) so that the person may grab the distal end 48 while still wearing gloves or mittens to disengage the latch 34. In this manner, the participant does not need to take off his/her gloves or mittens and expose his/her fingers to the cold air while putting on and adjusting the goggle 10. This also saves time in adjusting the strap tension during participation of the sport since the participant does not need to take time to remove his/her gloves to adjust the strap tension.

In use, the goggle may be provided to the participant as shown in FIG. 3. The first strap 12 is not engaged to the second strap 14. The participant may feed the first strap 12 through the attachment member 16 as shown by the dash lines in FIG. 2. Initially, the participant may engage the hooks 30 to the loops 32 so that the straps 12, 14 loosely fit around the participant's head. The participant then positions the goggle on the participant's head, and pulls the hooks 30 off of the loops 32, the participant pulls on the distal end 48 of the first strap 12 in the direction of arrow 50 to feed more of the first strap 12 through the attachment member 16. This increases the tension in the first and second straps 12, 14. The elastic portion(s) of the first and/or second straps 12, 14 stretch out to apply tension to the person's head so that the goggle 10 remains stationary on the person's head. When the tension in the first and second straps 12, 14 reaches the desired level, the participant pivots the end portion 52 of the first strap 12 in the direction of arrow 54 to engage the hooks 30 to the loops 32. If tension in the straps 12, 14 is too tight, then the hooks 30 may be released from the loops 32 to let some of the first strap 12 backtrack through the attachment member 16. When the tension in the first and second straps 12, 14 reaches the appropriate level, the participant then reengages the hooks and loops 30, 32. Simply put, the participant has adjusted the tension in the first and second straps 12, 14 while still wearing the goggle 10. No trial and error is required in order to set the appropriate tension in the first and second straps 12, 14. The participant did not remove the goggle from his/her head to adjust the tension in the straps. Rather, the participant pulls the first strap to adjust the strap tension and engages the latch mechanism to set the tension while still wearing the goggle. Moreover, the participant did not have to remove his/her gloves or expose his/her hands to the cold air in order to adjust the tension in the first and second straps 12, 14. Accordingly, the tension adjustment of the first and second straps 12, 14 may be accomplished while outside in the cold environment during the outdoor activity in a quick and convenient fashion. After the tension of the straps 12, 14 is adjusted, the participant may now place his/her helmet over the straps 12, 14. Since the straps 12, 14 are disposed under the helmet, the attachment member 16 may be fabricated from a semi rigid material so that the attachment member 16 does not hurt the participant's head in the event of an accident.

The goggle discussed herein was described as being an under the helmet version. The goggle is placed directly on the person's head or over a beanie. However, it is also contemplated that the goggle 10 may also be worn as an over the helmet goggle. In particular, the participant wears his/her helmet first and the lengths of the straps 12, 14 are adjusted. When the goggle is placed over the participant's eyes, the straps 12, 14 are disposed on the exterior surface of the helmet. Similar to the under the helmet version discussed above, the straps 12, 14 are may initially be adjusted so as to be loosely fitted around the helmet. Once the goggle 10 is placed on the participant's helmet, the participant may disengage the hooks 30 from the loops 32 to adjust the tension in the first and second straps 12, 14. The participant pulls the distal end 48 of the first strap 12 in the direction of arrow 50 and when the tension of the first and second straps 12, 14 have reached the desired level while wearing the goggle and helmet, the participant pivots the distal end portion 52 of the first strap 12 in the direction of arrow 54 to engage the hooks 30 through the loops 32. Once again the tension in the first and second straps 12, 14 is adjusted without trial and error and the participant does not have to remove his/her gloves or mittens and expose his/her hands to the cold air.

The sports goggle 10 disclosed herein was discussed in relation to a snow activity such as snow skiing, snowboarding or motorcycle riding. However, the goggle may have other applications such as use in a medical environment, manufacturing environment, chemical environment, etc.

Although the various aspects of the straps are discussed in relation to goggle frames having pivotable arms (see FIG. 1) and goggles that directly receive the straps (see FIG. 4), those of ordinary skill in the art will recognize that the various aspects of the straps discussed herein are also applicable to fixed outriggers that receive the straps as well as any other systems that directly or indirectly secure the straps to the frame of the goggle known in the art or developed in the future.

The above description is given by way of example, and not limitation. Given the above disclosure, one skilled in the art could devise variations that are within the scope and spirit of the invention disclosed herein, including various ways of attaching the first and second straps 12, 14 to the frame 18 of the goggle 10. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A sports goggle for viewing a person's surroundings and preventing particulate matter from contacting the person's eyes during an activity, the goggle comprising:
a lens disposable in front of the person's eyes;
a frame for holding the lens and having a soft cushion which interfaces with the person's face, the frame defining opposed left and right end portions;
a first strap directly or indirectly attached to one of the left or right end portions of the frame, a distal end portion of the first strap having first and second parts of a latch; and
a second strap directly or indirectly attached to the other one of left or right end portions of the frame, a distal end portion of the second strap having an attachment member;
wherein the distal end portion of the first strap is fed through the attachment member and the first part of the latch is engaged to the second part of the latch for securing the second strap and the first strap together at a fixed tension.

2. The goggle of claim 1 wherein at least a portion of the first strap is elastic, at least a portion of the second strap is elastic, or at least a portion of both the first and second straps are elastic.

3. The goggle of claim 1 wherein the first and second parts of the latch are hooks and loops, loops and hooks snaps or a button/button holes.

4. The goggle of claim 3 wherein the hooks and loops are disposed on an outer side of the second strap.

5. The goggle of claim 1 wherein the lens comprises left and right lenses.

6. The goggle of claim 1 wherein the first strap is directly attached to one of the left or right end portions of the frame with a first slot formed in the one of the left and right end portions for receiving and securing the first strap; and a second strap is directly attached to the other one of left or right end portions of the frame with a second slot formed in the other one of the left and right end portions for receiving and securing the second strap.

7. The goggle of claim 1 wherein the first strap is indirectly attached to one of the left or right end portions of the frame with a first outrigger movably attached to the one of the left or right end portions wherein the first strap is secured to the first outrigger; and a second strap is indirectly attached to the other one of left or right end portions of the frame with a second outrigger movably attached to the other one of the left or right end portions wherein the second strap is secured to the second outrigger.

8. The goggle of claim 1 wherein the attachment member is a ring or buckle.

9. A method for securing a goggle to a person's head, the method comprising the steps of:
providing a goggle with a first strap directly or indirectly attached to one of a left or right end portion of a goggle frame and a second strap directly or indirectly attached to the other one of the left or right end portion of the goggle frame wherein the first strap is fed through an attachment member attached to the second strap for adjusting tension in the first strap;
adjusting tension in the first strap by feeding more or less of the first strap through the attachment member; and
engaging a first part of a latch on the first strap to a second part of the latch on the first strap for securing the first strap to the attachment member.

10. The method of claim 9 further comprising the step of disposing the goggle around the person's head.

11. The method of claim 10 further comprising the step of disposing a helmet on the person's head over the first strap.

12. The method of claim 9 wherein the adjustment step comprises the step of pulling the first strap through the attachment member until tension in the first strap is at the desired level while the goggle is being worn on the person's head.
